(19) United States
(12) Patent Application Publication
(10) Pub. No.: US 2012/0177710 A1
(43) Pub. Date: Jul. 12, 2012

(54) TOPICALLY APPLICABLE COMPOSITION FOR USE AS A SKIN BLEACHING AGENT

(75) Inventors: Hugo ZIEGLER, Witterswil (CH); Peter Wikstroem, Gipf-Oberfrick (CH); Martin Stöckli, Burg im Liestal (CH)

(73) Assignee: DSM IP ASSETS B.V., Heerlen (NL)

(21) Appl. No.: 13/425,797

(22) Filed: Mar. 21, 2012

Related U.S. Application Data
(62) Division of application No. 12/089,654, filed on Aug. 28, 2008.

(30) Foreign Application Priority Data
Oct. 12, 2005 (CH) 1656/05

(51) Int. Cl. A61K 8/46; A61Q 17/04; A61K 8/11; A61K 8/14; A61Q 19/02; B82Y 99/00

(52) U.S. Cl. 424/401; 424/62; 424/59; 977/788; 977/926

(57) ABSTRACT

Topical composition for use especially as a skin lightener, characterized in that it contains an effective amount of at least one compound of general formula (I) or a mixture of such compounds and/or an acid addition salt thereof:

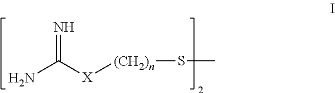

in which

X is NH or a direct bond and n is 2, 3 or 4, preferably 2 or 3, and its use for lightening skin colour, for depigmenting liver spots and for evening out non-uniformities in skin colouration. The invention further relates to dermatologically effective compositions containing at least one disulfide of general formula (I) and at least one additional skin care ingredient.

### CROSS-REFERENCE

This application is a divisional of commonly owned copending U.S. application Ser. No. 12/089,654, filed Aug. 28, 2008, which is the national phase application under 35 USC §371 of PCT/CH2006/000555, filed Oct. 9, 2006 which designated the US and claims benefit of CH 1656/05, filed Oct. 12, 2005, the entire contents of each of which are hereby incorporated by reference.

### FIELD OF THE INVENTION

The present invention relates to topical agents or compositions, especially skin lighteners or skin-lightening compositions, containing amidinoalkyl or guanidine-alkyl disulfides, and to the use of these compounds and the compositions according to the invention for lightening skin colour, for depigmenting liver spots and for evening out non-uniformities in skin colouration.

### BACKGROUND OF THE INVENTION

In many regions of the world the concept of beauty is directly associated with a light skin colouration. As a consequence of greater life expectancy and increasing UV exposure due to environmental pollution, more and more people are developing liver spots and pigmental moles. If these melanin-forming melanocytes are not uniformly distributed over human skin, patches are produced which are either lighter or darker in colour than the surrounding areas of skin. To alleviate this problem, skin lighteners are available on the market which help at least partially to even out such pigmental moles.

Skin-lightening substances intervene in some form or another in the melanin metabolism or catabolism. The melanins, which are normally brown to black in colour, are formed in the skin's melanocytes, transferred to the keratinocytes and cause the colouration of the skin or hair. In mammals the brown-black eumelanins are formed mainly of hydroxy-substituted aromatic amino acids like L-tyrosine and L-DOPA, and the yellow to red pheomelanins are additionally formed of sulfur-containing molecules like cysteine (Cosmetics & Toiletries 1996, 111(5), 43-51). The copper-containing key enzyme tyrosinase converts L-tyrosine to L-3,4-dihydroxyphenylalanine (L-DOPA), which in turn is oxidized, again by tyrosinase, to melanin via dopaquinone, which is red-brown in colour. A comparison of tyrosinases from plants, fungi and mammalian cells shows that the mechanism and the substrate specificity are comparable for all the tyrosinases studied. Skin-lightening substances are known per se, but do not satisfy users' demands. In particular, hydroquinone, azelaic acid and kojic acid carry a high toxicological risk and are banned in some countries. Arbutins and arbutin derivatives, as well as vitamins and vitamin derivatives, are unsatisfactory in terms of their stability. Glabridine, undecylenoylphenylalanines, hexapeptides 2 and Broussonetia extract powder have to be used in relatively high doses to effect satisfactory skin bleaching.

It is known that cystamine [bis(2-aminoethyl)disulfide], as a non-toxic active substance, is capable of depigmenting human melanoma cells, as well as normal melanocytes, effectively and reversibly (J. Invest. Dermatol, 2000, 21-27). The basis of the action of cystamine is that in a first step it is reduced to cysteamine, which then undergoes reactions with products having tyrosinase activity, thereby preventing the synthesis of melanin, especially the brown/black eumelanin. The object of the present invention is to find active substances which are inexpensive, easy to prepare, highly effective and stable and do not inhibit tyrosinase, and which can be used in skin lighteners. It has now been found that replacement of the amino groups of cystamine with amidino and guanidino radicals is capable of increasing the depigmenting action to a surprising extent. The compounds of general formula (I) set a new standard in skin lightening and the treatment of pigmental moles.

### Description, General Section

The present invention relates to a topical agent or topical composition for use especially as a skin lightener or skin-lightening composition and for bleaching liver spots and pigmental moles, characterized in that it contains an effective amount of at least one compound of general formula (I) or a mixture of such compounds and/or an acid addition salt thereof:

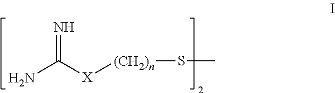

in which
X is NH or a direct bond and n is 2, 3 or 4, preferably 2 or 3.

The invention further relates to the use of the topical agent or topical composition according to the invention, containing an effective amount of at least one compound of general formula (I) or a mixture of such compounds and/or an acid addition salt of such compounds, as a skin lightener or skin-lightening composition, for bleaching liver spots and pigmental moles and/or for evening out non-uniformities in skin colouration.

The invention further relates to the use of a compound of general formula (I) above or a mixture of such compounds and/or an acid addition salt thereof for the preparation of a topical agent, or topical composition for use especially as a skin lightener or skin-lightening composition, for bleaching liver spots and pigmental moles and/or for evening out non-uniformities in skin colouration.

The compounds of formula (I) can form pure or mixed monobasic or dibasic salts with acids, e.g. with inorganic acids such as hydrochloric acid, hydrobromic acid, sulfuric acid or phosphoric acid; with suitable organic saturated or unsaturated aliphatic carboxylic acids, e.g. aliphatic monocarboxylic or dicarboxylic acids such as formic acid, acetic acid, trifluoroacetic acid, trichloroacetic acid, propionic acid, glycolic acid, succinic acid, fumaric acid, malonic acid, maleic acid, oxalic acid, phthalic acid, citric acid, lactic acid or tartaric acid; with aromatic carboxylic acids such as benzoic acid or salicylic acid; with aromatic-aliphatic carboxylic acids such as mandelic acid or cinnamic acid; with heteroaromatic carboxylic acids such as nicotinic acid; with aliphatic or aromatic sulfonic acids such as methanesulfonic acid or toluenesulfonic acid; or with ascorbic acid. Dermatologically acceptable salts are preferred.

Preferred compounds of formula (I) are those in which n=2, and their pure dibasic acid addition salts.

The preparation of the compounds of general formula (I) is known per se. Thus the guanidino compounds are obtainable on the one hand by amidation of the corresponding amino derivatives, e.g. as described in British Journal of Pharmacology 118, 1659-1668 (1996), or on the other hand by the rearrangement and subsequent oxidation of aminoalkylisothiouronium salts [Chem. Pharm. Bull. 14(11), 1193-1201 (1966)]. The amidino derivatives can be prepared by reacting the cyanoalkyl disulfides, e.g. as described in WO-03/072559 and according to the literature cited therein.

The topical agents or compositions according to the invention can contain the compound of formula (I) or a mixture of these compounds and/or their salts in concentrations in the range between 0.005 and 50% by weight, preferably between 0.05 and 5% by weight, based on the weight of the agents or compositions, or in concentrations varying within this range.

The topical agents or compositions according to the invention can be used in the form of a solution, a dispersion or an emulsion, encapsulated in an excipient, such as macro-, micro- or nanocapsules, or in liposomes or chylomicra, included in macro-, micro- or nanoparticles or in microsponges, or absorbed on pulverulent organic polymers or mineral excipients, such as talcum, bentonite or other mineral excipients.

The topical agents or compositions according to the invention can be used in any galenical form. Examples of such forms are W/O and O/W emulsions, milks, lotions, ointments, gelling and viscous, surface-active and emulsifying polymers, pomades, shampoos, soaps, gels, powders, sticks, sprays, body oils, face masks and plasters.

The topical agents or compositions according to the invention, especially skin lighteners, can contain cosmetic auxiliary substances and additives to form compositions like those conventionally used in such formulations, examples being sunscreens (e.g. organic or inorganic light filters, preferably micropigments), preservatives, bactericides, fungicides, virucides, cooling agents, plant extracts, e.g. Scutellaria extract, Saxifrage extract, peptides and derivatives thereof, enzyme inhibitors, anti-inflammatories, astringents, e.g. aluminium chlorohydrate and aluminium zirconium tetra chlorohydrex glycine, substances that accelerate wound healing (e.g. chitin or chitosan and derivatives thereof), film-forming substances (e.g. polyvinylpyrrolidones or chitosan or derivatives thereof), common anti-oxidants, vitamins (e.g. vitamin C and derivatives, tocopherols and derivatives, vitamin A and derivatives), 2-hydroxycarboxylic acids (e.g. citric acid, glycolic acid, malic acid, L-, D- or di-lactic acid), perfumes, antifoams, dyestuffs, pigments with a colouring action, thickeners such as acrylate/C10-30 alkyl acrylate crosspolymers, polyacrylamides, cationic polymers, gums (e.g. xanthan gum, guar gum) and cellulose derivatives (such as hydroxyethyl cellulose, hydroxypropyl cellulose), propellant gases, e.g. propane, butane, isobutane, dimethyl ether or carbon dioxide, penetration improvers (e.g. ethoxy diglycol), surfactants, emulsifiers, plasticizers, moisturizers and/or humectants (e.g. glycerol or urea), fats, oils, waxes, unsaturated fatty acids or derivatives thereof (e.g. linoleic acid, alpha-linolenic acid, gamma-linolenic acid or arachidonic acid and their respective natural or synthetic esters), or other conventional constituents of a cosmetic or dermatological formulation, such as water, alcohols or polyols, e.g. propylene glycol, polyethylene glycol, polypropylene glycol, glycerol, 1,2,4-butanetriol, 1,2,6-hexanetriol, ethanol or isopropanol, sorbitol esters, butanediol, acetone, ethylene glycol monoethyl ether, diethylene glycol monobutyl ether, diethylene glycol monoethyl ether, skin moisturizers, such as glycerol, diglycerol, triglycerol, polyglycerol, ethoxylated and propoxylated glycerol, polypropylene glycol, polyethylene glycol, ethylene glycol, diethylene glycol, triethylene glycol, propylene glycol, dipropylene glycol, hexylene glycol, 1,3-butylene glycol or 1,4-butylene glycol, polymers, foam stabilizers, electrolytes, organic solvents, silicone derivatives or chelating agents (e.g. ethylenediaminetetraacetic acid and derivatives).

By simple trial and error, those skilled in the art can easily determine the amounts of cosmetic or dermatological auxiliary substances and additives and perfume to be used in each case according to the particular type of product.

Preferably, the compositions according to the invention can also contain other skin-lightening substances, in particular, the skin lighteners according to the invention can also contain kojic acid, kojic acid derivatives, niacin/niacinamides, alpha-hydroxycarboxylic acids, such as lactic acid, arbutin, arbutin derivatives, ascorbic acid, ascorbic acid derivatives, such as sodium ascorbyl phosphate, magnesium ascorbyl phosphate and ascorbyl glucoside, hydroquinone, hydroquinone derivatives, glabridin in liquorice, oleanolic acid, sulfur-containing molecules, e.g. glutathione or cysteine, or other synthetic or natural skin lightening substances, it also being possible for the latter to be used in the form of a plant extract, e.g. bearberry extract, mulberry extract or rice extract.

Particularly preferably, the compositions according to the invention can contain inorganic or organic sunscreens or UV blockers. Inorganic sunscreens which can be used here include the following metal oxides: titanium dioxide, zinc oxide, zirconium oxide and iron oxide, and mixtures thereof.

Examples of organic sunscreens are dicamphorsulfonic acid (Mexoryl SX), drometrizole trisiloxane (Mexoryl XL), 2-ethylhexyl p-methoxycinnamate (commercially available as PARSOL MCX), 4,4′-t-butylmethoxydibenzoylmethane (commercially available as PARSOL 1789), 2-hydroxy-4-methoxybenzophenone, octyldimethyl-p-aminobenzoic acid, digalloyl trioleate, 2,2-dihydroxy-4-methoxybenzophenone, ethyl 4-(bis(hydroxypropyl))aminobenzoate, 2-ethylhexyl 2-cyano-3,3-diphenylacrylate, 2-ethylhexyl salicylate, glyceryl p-aminobenzoate, 3,3,5-trimethylcyclohexyl salicylate, methyl anthranilate, p-dimethylaminobenzoic acid or p-dimethylaminobenzoate, 2-ethylhexyl p-dimethylaminobenzoate, 2-phenyl-benzimidazole-5-sulfonic acid, 2-(p-dimethylaminophenyl)-5-sulfonic benzoxazoic acid, octocrylene, 2,2′-methylene-bis[6-(2H-benzotriazol-2-yl)-4-(1,1,3,3-tetra-methylbutyl)phenol] obtainable from Ciba SC as TINOSORB™ M, 2,4-bis[(4-(2-ethylhexyloxy)-2-hydroxy)phenyl]-6-(4-methoxyphenyl)-1,3,5-triazine obtainable from Ciba SC as TINOSORB™ S, and mixtures of these compounds.

Also of particular use in the compositions are sunscreens such as those disclosed in U.S. Pat. No. 4,937,370, granted to Sabatelli on 26 Jun. 1990, and U.S. Pat. No. 4,999,186, granted to Sabatelli & Spirnak on 12 Mar. 1991. The sunscreens disclosed in said patent documents have two inherent chromophoric groups in a single molecule which exhibit different ultraviolet absorption spectra. One of the chromophoric groups absorbs predominantly in the UV-B region and the other absorbs strongly in the UV-A region. Preferred compounds in this class of sunscreens are 4-N,N-(2-ethylhexyl)methylaminobenzoic acid esters of 2,4-dihydroxybenzophenone, N,N-di(2-ethylhexyl)-4-aminobenzoic acid esters of 4-hydroxydibenzoylmethane, 4-N,N-(2-ethylhexyl)methylaminobenzoic acid esters of 4-hydroxydibenzoylmethane, 4-N,N-(2-ethylhexyl)methylaminobenzoic acid esters of 2-hydroxy-4-(2-hydroxyethoxy)benzophenone, 4-N,N-(2-ethylhexyl)methylaminobenzoic acid esters of 4-(2-hydroxyethoxy)dibenzoylmethane, N,N-di(2-ethylhexyl)-4-aminobenzoic acid esters of 2-hydroxy-4-(2-hydroxyethoxy)benzophenone and N,N-di(2-ethylhexyl)-4-aminobenzoic acid esters of 4-(2-hydroxyethoxy)dibenzoylmethane, and mixtures thereof. The exact amounts depend on the chosen sunscreen(s) and the desired light protection factor (LPF).

The topical compositions according to the invention also contain a dermatologically acceptable excipient. The expression “dermatologically acceptable excipient” is understood here as meaning that the excipient is suitable for topical application to the horny tissue, has good aesthetic properties, is compatible with the active substances of the present invention and any other components, and does not give rise to unfavourable safety or toxicity considerations.

The excipient can take many different forms, for example, it is possible here to use emulsion excipients, including oil-in-water, water-in-oil, water-in-oil-in-water and oil-in-water-in-silicone emulsions.
A) Water-in-silicone emulsions
   Water-in-silicone emulsions contain a silicone continuous phase and an aqueous disperse phase.
B) Oil-in-water emulsions

Other preferred topical excipients include oil-in-water emulsions with an aqueous continuous phase and a hydrophobic, water-insoluble phase (“oil phase”) dispersed therein. Examples of suitable oil-in-water emulsion excipients are described in U.S. Pat. No. 5,073,371 to D. J. Turner et al. granted on 17 Dec. 1991, and U.S. Pat. No. 5,073,372 to D. J. Turner et al., granted on 17 Dec. 1991.

### EXPERIMENTAL SECTION

### Abbreviations and Terms Used

SDS sodium dodecylsulfate
EDTA ethylenediaminetetraacetic acid
NaHCO₃ sodium hydrogen carbonate
PBS phosphate buffered saline
Proteinase K from Tritirachlum album
HCl hydrogen chloride
HBr hydrogen bromide

### Example 1

### Preparation of a Lotion

A lotion was prepared by conventional methods according to the following formulation (data in % by weight):

| | | | % by |
|---|---|---|---|
| Phase | Ingredients | INCI name | weight |
| A | Pationic 138C | Sodium Lauroyl Lactylate | 0.34 |
| | Cetyl alcohol | Cetyl Alcohol | 2.00 |
| | Tegin 4100 | Glyceryl Stearate | 2.00 |
| | Tegosoft TN | C12-15 Alkyl Benzoate | 7.00 |
| | Tegosoft CT | Caprylic/Capric Triglycerides | 7.00 |
| B | Water | Aqua | 75.96 |
| | Propylene glycol | Propylene Glycol | 5.00 |
| | Preservatives | | q.s. |
| | Keltrol RD | Xanthan Gum | 0.20 |
| C | Citric acid solution | Citric Acid | q.s. |
| D | Compound 1 | | 0.50 |

Heat phases A and B separately to 75° C., add phase A to phase B, with stirring, and homogenize, allow to cool to room temperature, then adjust the pH to approx. 5.0-5.5 with phase C and add phase D, with stirring.

### Example 2

### Preparation of a Cream

A cream was prepared by conventional methods according to the following formulation (data in % by weight):

| | | | % by |
|---|---|---|---|
| Phase | Ingredients | INCI name | weight |
| A | Water | Aqua | 67.10 |
| | Glycerol | Glycerin | 5.00 |
| | Preservatives | | q.s. |
| B | Crodafos CES | Cetearyl Alcohol (and) Dicetyl | 5.00 |
| | | Phosphate (and) Ceteth-10 Phosphate | |
| | Myritol 331 | Coco Glyceride | 6.00 |
| | Tegosoft TN | C12-15 Alkyl Benzoate | 3.00 |
| | Tegosoft DC | Decyl Cocoate | 3.00 |
| | Fitoderme | Squalane | 2.00 |
| C | NaOH solution | Sodium Hydroxide | q.s. |
| D | Dow Corning 345 | Cyclomethicone | 3.00 |
| | Aristoflex AVC | Ammonium Acryloyldimethyltaurate/ | 0.40 |
| | | VP Copolymer | |
| E | EDG Plus | Ethoxydiglycol | 5.00 |
| | Compound 1 | | 0.50 |

Heat phases A and B separately to 75° C., add phase B to phase A, with stirring, and homogenize, adjust the pH to approx. 5.5-6.5 with phase C, successively add the components of phase D at approx. 65° C. and homogenize. Successively add the components of phase E at approx. 35° C., cool to room temperature and, if necessary, readjust the pH to 5.0-5.5.

### Example 3

### Preparation of a Sun Cream

A sun cream was prepared by conventional methods according to the following formulation (data in % by weight):

| Phase | Ingredients | INCI name | % |
|---|---|---|---|
| A | Amphisol K | Potassium Cetyl Phosphate | 2.00 |
| | Tegosoft CT | Caprylic/Capric Triglycerides | 4.00 |
| | Tegosoft TN | C12-15 Alkyl Benzoate | 4.00 |
| | Cetiol SN | Cetearyl Isononanoate | 1.00 |
| | Tegin | Glyceryl Stearate | 3.00 |
| | Cetyl alcohol | Cetyl Alcohol | 1.00 |
| | Uvinul T 150 | Ethylhexyl Triazone | 0.50 |
| | Tinosorb OMC | Ethylhexyl Methoxycinnamate | 3.00 |
| B | Water | Aqua | 64.75 |
| | Glycerol | Glycerin | 3.00 |
| | Keltrol RD | Xanthan Gum | 0.25 |
| C | Tinosorb M | Methylene Bis-Benzotriazolyl | 8.00 |
| | | Tetramethylbutylphenol | |
| D | EDG Plus | Ethoxydiglycol | 5.00 |
| | Preservatives | | q.s. |
| | Compound 1 | | 0.50 |
| E | Citric acid solution | Citric acid | q.s. |

Heat phases A and B separately to 75° C., add phase A to phase B, with stirring, and homogenize, adjust the pH of phase C to approx. 5.5, allow to cool to 40° C., then successively add phase C and the components of phase D and adjust the pH to 4.5-5.0 with phase E.

### Example 4

### Determination of the Reduction in Melanin Content after the Treatment of Melanoderm Tissues of the Dark Skin Type with Test Substances

Materials and methods

### MelanoDerm Mel-300B Lot 6715 Kit G

Long life maintenance medium

### 1% SDS in 10 mM Tris, 0.05 mM EDTA pH 6.8

Proteinase K 5 mg/ml in 1% SDS in 10 mM Tris, 0.05 mM EDTA pH 6.8

### 500 mM NaHCO₃

### Chloroform/methanol (2:1)

Test substances: compound 1 and cystamine as standard
Cultivation of the Tissues and Application of the Test Substances

The tissues, treated according to the manufacturer's instructions, were transferred to 6-well plates containing 3 ml of medium per well. The tissues were placed on membrane inserts (Transwell-Clear from Corning). 2 ml of medium were pipetted into the lower compartment and 1 ml of medium into the upper compartment. The test substances were applied topically as an aqueous solution at a rate of 10 mM per tissue. The medium and the test substance were renewed every 48 hours by rinsing the surface of the tissues with 500 μl of warm PBS and applying fresh test substance. After 8 days the test substance was removed from two tissues per treatment type and cultivation was continued for a further eight days with normal medium not containing test substance. The other two tissues per treatment type were incubated further with test substance. The experiment lasted a total of 16 days.
Extraction of the Melanin

The membranes with the pigmented epidermis were cut out of the holders with a scalpel and transferred to screw-capped Eppendorf tubes. 200 μl of 1% SDS in 10 mM Tris, 0.05 mM EDTA pH 6.8 and 20 μl of Proteinase K were pipetted into each tube. The Eppendorf tubes were incubated overnight in a Thermomixer at 45° C. The following morning a further 20 μl of Proteinase K were pipetted into each tube and incubation was continued for 6 hours. The lysate was rendered basic by the addition of 25 μl of 500 mM NaHCO₃ and incubated in the Thermomixer for 30 min at 80° C. Fats and proteins were removed from the lysate by treatment with 200 μl of methanol/chloroform (2:1). 3×100 μl of supernatant were pipetted into a 96-well plate and the absorption was measured at 450 nm.
Results

Melanin content in %

| | after 16 days | 8 days of treatment, |
|---|---|---|
| Test substance | of treatment | measured after 16 days |
| Control | 100 | — |
| Water | 85 | 85 |
| Compound 1 | 12 | 23 |
| Cystamine (standard) | 30 | 52 |

The following Table contains preferred individual compounds of general formula I:

| Compound | | |
|---|---|---|
| no. | Structural formula | Acid addition salts |
| 1 2 3 4 | 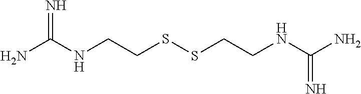 | × 2 HBr × 2 ascorbate × 2 AcOH — |
| 5 6 7 8 | 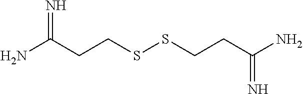 | × 2 HCl × 2 lactate × 2 trifluoroacetate — |
| 9 10 11 12 | 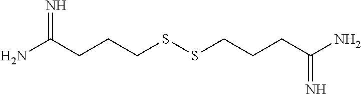 | × 2 ascorbate × 2 HCl × 2 AcOH — |
| 13 14 15 16 | 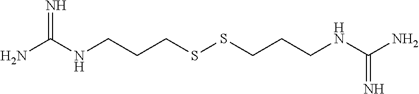 | × 2 HBr × 2 sulfate × 2 salicylate — |

What is claimed is:

1. A method of lightening skin comprising topically applying to skin in need of lightening a composition which comprises an effective amount of at least one compound of the formula: 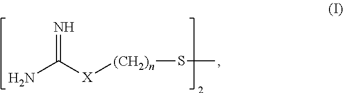
wherein —X— is —N(H)—, or a direct bond, and n is 2, 3, or 4, in free form or in acid addition salt form.

2. The method of claim 1, wherein the composition is in the form of an emulsion; a milk, a lotion, a cream, an ointment, a gelling and viscous, surface-active and emulsifying polymer; a pomade, a shampoo, a soap, a gel, a powder, a stick, a spray, a body oil, a face mask, or a plaster.

3. The method of claim 1, wherein the formula (I), n is 2, or 3.

4. The method of claim 3, wherein the formula (I), —X— is —N(H)—, and n is 2.

5. The method of claim 1, wherein the composition comprises between 0.005 and 50% by weight of the at least one compound of the formula I.

6. The method of claim 5, wherein the composition comprises between 0.05 and 5% by weight of the at least one compound of the formula I.

7. The method of claim 1, wherein the composition comprises:
(a) in the form of a solution, a dispersion, or an emulsion, or
(b) encapsulated in an excipient, or
(c) in a liposome or a chylomicron, or
(d) included in a macro-, micro- or nanoparticle, or a microsponge, or
(e) absorbed on a pulverulent organic polymer, or a mineral excipient.

8. The method as in claim 1, wherein the composition is present in the form of a water-in-oil emulsion, or an oil-in-water emulsion.

9. The method as in claim 1, wherein the composition is present in the form of a macro-, micro- or nanocapsule.

10. The method as in claim 1, wherein the composition further comprises:
a) a safe effective amount of at least one additional skin care active ingredient, selected from the group consisting of a skin bleaching ingredient, a depigmentation ingredient, a sun protection ingredient, and a UV blocking ingredient, and
b) optionally a dermatologically acceptable excipient.
